(19) United States
(12) Patent Application Publication
(10) Pub. No.: US 2011/0268812 A1
(43) Pub. Date: Nov. 3, 2011

(54) Cosmetic or dermatological composition comprising the combination of honey and a peptide

(75) Inventors: Jean-Hubert Cauchard, Orleans (FR); Frëdëric Bonte, Orleans (FR); Emmanuelle Noblesse, Donnery (FR); Carine Nizard, Ivry Sur Seine (FR)

(73) Assignee: LVMH RECHERCHE, Saint Jean de Braye (FR)

(21) Appl. No.: 12/932,449

(22) Filed: Feb. 25, 2011

(30) Foreign Application Priority Data
Mar. 9, 2010 (FR) 1051708

(51) Int. Cl. A61K 8/98; A61Q 19/08

(52) U.S. Cl. 424/537

(57) ABSTRACT

A cosmetic composition has a combination of honey or royal jelly, and a peptide, including at least the amino acid sequence Gly-X₁-X₂-Pro-Gly. Applying the composition makes it possible to delay the appearance of the signs of skin aging or to slow down the effects thereof.

This application claims the benefit of Serial No. 1051708, filed 9 Mar. 2010 in France and which application is incorporated herein by reference. A claim of priority, to the extent appropriate is made.

The subject of the invention is a cosmetic composition comprising the combination of honey, or royal jelly, and a peptide, comprising at least the amino acid sequence Gly-X₁-X₂-Pro-Gly.

### PRIOR ART

As is well known, skin aging is the result of genetic factors, but also environmental factors such as, for example, exposure to the sun, and manifests itself at the same time through molecular, cellular, histological and clinical modifications at the level of the epidermis and the dermis.

Among these skin-aging-related modifications, a decrease is observed in the thickness of the epidermis and in the size of the epidermal ridges, inducing a flattening of the dermal-epidermal junction which produces less cohesion at the interface of the epidermis and the dermis. A decrease in fibroblast density is also observed in the dermis.

This skin atrophy is also gradually reflected in changes to the extracellular matrix, linked to a decrease in the amount of collagen produced and to a decline in the levels of proteoglycans, glycosaminoglycans and fibronectin which are constitutive elements of said extracellular matrix, and in the levels of elastin fibres in the skin.

These phenomena lead to fragility of the skin, impairment of its mechanical and functional properties and of its protective and barrier functions, and, finally, a loss of elasticity which makes the expression wrinkles more visible.

Conversely, healing is itself a tissue repair process which comprises several steps, including a step of repair of the dermis and of the epidermis, resulting in re-epithelialisation of the wound (Ann. Dermatol. Venereol. 2005, 132:8549-68). The tissue repair phase corresponds to fibroblast proliferation and to extracellular matrix synthesis. The epithelialisation phase comprises the migration of epithelial cells (keratinocytes), and then the differentiation thereof so as to reform the epidermis. The extracellular matrix is gradually remodeled.

The main defect of skin which heals and repairs itself alone is often its lack of plasticity and elasticity. This is also true for skin which ages or which has been exposed to the sun (photoaging/elastosis). However, this quality is essential to the biomechanical properties and the beauty of the skin.

### OBJECTS OF THE INVENTION

The main purpose of the present invention is to provide a novel cosmetic agent, which can in particular be used in the context of a cosmetic composition, capable of accelerating tissue repair processes, by stimulating the fibroblasts which behave as real skin construction accelerators, so as to result in a tissue repair process that acts as an internal-growth and matrix-reorganization factor, for a more dense and more elastic dermis.

Another main purpose of the invention is to provide a cosmetic composition comprising such a cosmetic agent.

Another main purpose of the invention is to provide a cosmetic care method which comprises the application of such a cosmetic agent.

### SUMMARY OF THE INVENTION

The inventors have presently discovered that a combination of honey, or royal jelly, and a peptide comprising a particular sequence makes it possible to accelerate tissue repair processes, by stimulating the fibroblasts which behave as real skin reconstruction accelerators.

The abovementioned combination thus allows a tissue repair process that acts as an internal-growth and matrix-reorganization factor for a more dense and more elastic dermis.

### BRIEF DESCRIPTION OF THE DRAWINGS

FIGS. 1a, 1b and 2a to 2d are examples illustrating the methodology used for demonstrating the activity of the combination of the invention.

FIGS. 1a and 1b are two examples of observations, made under a phase contrast microscope, of an artificially “damaged” zone of the well in which normal human fibroblasts (NHFs) are cultured. The image in FIG. 1a visualizes a zone devoid of cells, corresponding to the place where the IBIDI culture insert was located, immediately after withdrawal of said insert. FIG. 1b visualizes the same zone after partial recolonization by the NHFs.

FIGS. 2a to 2d represent the steps of the captured image analysis in blue fluorescence and in red fluorescence, making it possible to characterize the colonization, by the fibroblasts, of the zone of the support artificially damaged by means of the abovementioned culture inserts.

FIG. 3 represents, along the Y-axis, in the form of a bar chart, the number of cells having recolonized the scar per square centimetre, obtained with each of the products tested, respectively for the nontreated control, denoted NT; thyme honey and the Pal-VGVAPG peptide, each being used alone, as positive control, and, finally, the combination of clover honey and the abovementioned peptide, said combination constituting the invention.

### DETAILED DESCRIPTION OF THE INVENTION

A first subject of the present invention is thus directed towards a cosmetic agent, or a cosmetic composition comprising at least one cosmetically acceptable excipient, characterized in that it comprises honey or royal jelly, and a peptide, or a derivative of said peptide, comprising the sequence Gly-X₁-X₂-Pro-Gly, X₁ and X₂ being amino acids selected from natural or synthetic amino acids, and derivatives thereof. The present invention thus relates to a combination of honey or royal jelly, and a peptide, or a derivative of said peptide, comprising the sequence Gly-X₁-X₂-Pro-Gly and to a cosmetic composition comprising this combination.

The abovementioned peptide of the combination advantageously comprises 5 to 8 amino acids, advantageously selected from natural or synthetic amino acids, or a derivative of these amino acids.

X₁ and X₂ are preferably selected from valine (Val), proline (Pro), alanine (Ala), glycine (Gly), lysine (Lys), serine (Ser), aspartic acid (Asp), arginine (Arg) and isoleucine (Ile), or a salt or derivative thereof.

Among the preferred peptides, mention is made of those comprising or formed by a sequence selected from:

Val-Gly-Lys-Ser-Pro-Gly
Ile-Gly-Lys-Ser-Pro-Gly
Pro-Gly-Gly-Val-Lys-Pro-Gly
Val-Gly-Val-Val-Pro-Gly
Ile-Gly-Lys-Gly-Pro-Gly-Gly-Val
Val-Gly-Ala-Met-Pro-Gly
Val-Gly-Ala-Ser-Pro-Gly
Val-Gly-Lys-Met-Pro-Gly
Ile-Gly-Ala-Met-Pro-Gly
Ile-Gly-Ala-Ser-Pro-Gly
Ile-Gly-Lys-Met-Pro-Gly
Val-Gly-Val-Ala-Pro-Gly
Gly-Val-Ala-Pro-Gly-Val
Val-Gly-Val-Ala-Pro-Gly
Gly-Asp-Ser-Pro-Gly-Asp-Lys
Pro-Gly-Gly-Val-Leu-Pro-Gly
Val-Gly-Val-Val-Pro-Gly
Ile-Gly-Leu-Gly-Pro-Gly-Gly-Val
Val-Gly-Leu-Ser-Pro-Gly
Ile-Gly-Leu-Ser-Pro-Gly
Ile-Gly-Val-Ala-Pro-Gly
Val-Gly-Val-Ala-Pro-Gly
Gly-Ala-Ala-Pro-Gly
Gly-Val-Val-Pro-Gly
Gly-Gly-Gly-Pro-Gly
Gly-Leu-Leu-Pro-Gly
Gly-Ile-Ile-Pro-Gly
Gly-Ser-Ser-Pro-Gly
Gly-Thr-Thr-Pro-Gly
Gly-Cys-Cys-Pro-Gly
Gly-Met-Met-Pro-Gly
Gly-Phe-Phe-Pro-Gly
Gly-Tyr-Tyr-Pro-Gly
Gly-Trp-Trp-Pro-Gly
Gly-Asp-Asp-Pro-Gly
Gly-Asn-Asn-Pro-Gly
Gly-Glu-Glu-Pro-Gly
Gly-Gln-Gln-Pro-Gly
Gly-Arg-Arg-Pro-Gly
Gly-His-His-Pro-Gly
Gly-Lys-Lys-Pro-Gly
Gly-Pro-Pro-Pro-Gly
Gly-(3-hydroxyproline)-(3-hydroxyproline)-Pro-Gly
Gly-4-hydroxyproline-4-hydroxyproline-Pro-Gly
Gly-Gly-Gln-Gln-Pro-Gly-Leu
Ala-Val-Gly-Val-Ala-Pro-Gly
Ala-Val-Gly-Val-Ala-Pro-Gly-Leu
Val-Gly-Gly-Val-Pro-Gly
Leu-Gly-Thr-Ile-Pro-Gly.

The peptide of the combination can be advantageously esterified with at least one linear or branched, saturated or unsaturated, hydroxylated or nonhydroxylated, sulphur-containing or non-sulphur-containing fatty acid comprising from 2 to 22 carbon atoms.

According to one preferred embodiment, the peptide is esterified with a fatty acid comprising between 8 and 22 carbon atoms, that may advantageously be a palmitoyl, stearoyl, elaidoyl, lauroyl, myristoyl, stearoyl, oleoyl, arachidyl or linoleoyl group.

The particularly preferred peptide of the combination is a peptide comprising or formed by the sequence Val-Gly-Val-Ala-Pro-Gly, or a cosmetically acceptable salt, said peptide also being advantageously esterified with a palmitoyl or stearoyl group, or a cosmetically acceptable salt.

A preferred peptide is palmitoyl-Val-Gly-Val-Ala-Pro-Gly sold in the form of an aqueous-glycolic solution under the trade name Biopeptide EL by the company Sederma.

The honey may be a unifloral or polyfloral honey.

The honey is preferably a clover (Trifolium repens) honey, a thyme (Thymus vulgaris) honey or alternatively a manuka (Leptospernum scoparium) honey.

The cosmetic agent according to the present invention can be incorporated into a cosmetic composition.

Thus, the present invention is also directed towards a cosmetic composition comprising a cosmetic agent as defined above or as resulting form the following description.

The concentration of honey or royal jelly which is used as one of the two components of the cosmetic agent according to the invention could be

In the examples, all the percentages are given by weight, the temperature is in degrees Celsius, and the pressure is atmospheric pressure, unless otherwise indicated.

### Example 1

### Measurement of the Activity of the Combination of a Honey with a Peptide and/or a Peptide Derivative on Healing

This experiment is based on a model of biocompatible cell culture inserts allowing quantitative and perfectly reproducible measurement of the phenomena of healing in vitro.

1. Cell Culture

The cells used in this study are normal human fibroblasts (NHFs).

The cells are seeded in 175 cm² flasks at a density of 1 million per flask in DMEM (Dulbecco's Modified Eagle's Medium) culture medium supplemented with 10% of foetal calf serum and 1.3 mM L-glutamine. Having reached confluence, the cells are trypsinized and re-seeded for the healing test.

2. Healing Test

Seeding of the Cells

Before seeding of the cells, inserts sold in the form of kits under the name Culture-Insert (ref. 80209) by the company IBIDI (Germany) are placed at the bottom of six wells of a plate. The NHFs are seeded in a proportion of 20 000 cells/cm² in DMEM medium containing 10% of foetal calf serum, and cultured for 48 h.

When the cells have reached confluence, the culture medium is changed and replaced with DMEM alone. The culture is continued for 24 h.

Treatments

Two wells are used per treatment (substance tested) in this study.

The substances tested are the following (the concentrations indicated are the final concentrations):

| Clover honey | 100 | μg/mL |
|---|---|---|
| Pal-VGVAPG in aqueous-alcoholic solution | 1% | |
| Clover honey + Pal-VGVAPG | 100 | μg/mL + 1% |

A cell culture is also carried out in DMEM medium alone, without the addition of test substance, in two wells, in order to carry out a nontreated control (NT).

The Pal-VGVAPG peptide is in the form of an aqueous-glycolic solution, sold by the company Sederma under the trade name Biopeptide EL.

The active agents tested are prepared in the following way:

A stock solution at 100 mg/ml of clover honey in PBS is prepared and sterilized by filtration through a 0.22 μm filter. The final dilution ( 1/1000th) is then made in DMEM alone.

The commercial Biopeptide EL solution is diluted to 1/100th in the culture medium.

Healing

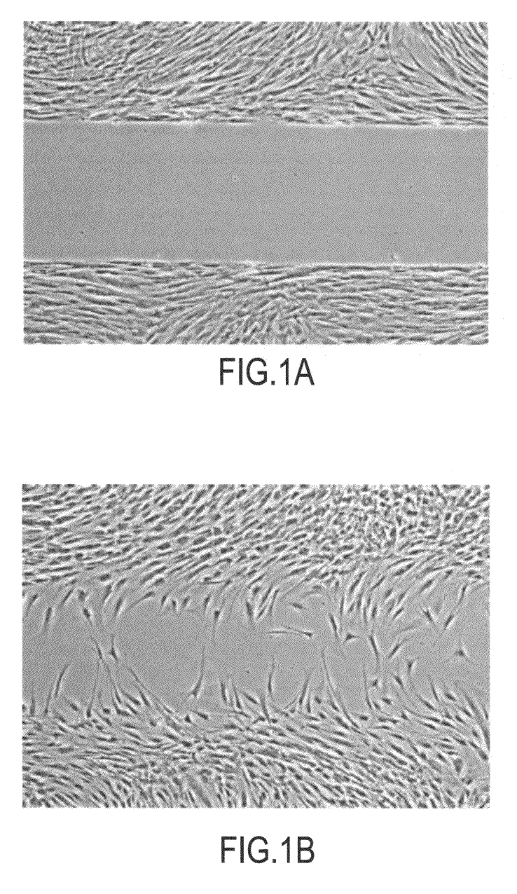

The culture insert is first of all removed with sterile tweezers. Using a phase contrast microscope, a “damaged” zone, devoid of any cells, is observed at the place where the insert was located (FIG. 1a).

The initial culture medium is then replaced in each well with the culture medium containing the active agents tested according to the conditions described above.

The cells are maintained in culture for 16 h (optimum measurement time determined previously), in an incubator at 37° C. and in an atmosphere containing 5% CO₂. The degree of recolonization of the cicatricial zone according to the treatments is observed by means of an image analysis technique (FIG. 1b).

Cell Labelling

After rinsing with PBS, the cells are fixed for 10 minutes with 10% formalin solution and then rinsed with PBS. The cells are then permeabilized with a 0.1% Triton X100 solution for 10 minutes.

A solution containing DAPI (′,6′-diamidino-2-phenylindole), a fluorescent molecule used for labelling cell nuclei, and phalloidin 546, used for labelling the actin cytoskeleton, is deposited onto the cells and incubated at ambient temperature, in the dark for 1 h.

The cells are then rinsed with PBS and covered with a drop of aqueous mounting medium.

Images are immediately taken on a Nikon TE2000 video microscope in blue fluorescence (cell nuclei) and red fluorescence (actin filaments), at a rate of 2 photos per well at the damaged zone.

Image Analysis

Image analysis is used to visualize and quantify the colonization by fibroblasts of the artificially “damaged” zone after removal of the insert. The Leica QWIN software is used for the analysis.

The analysis comprises the following four steps:

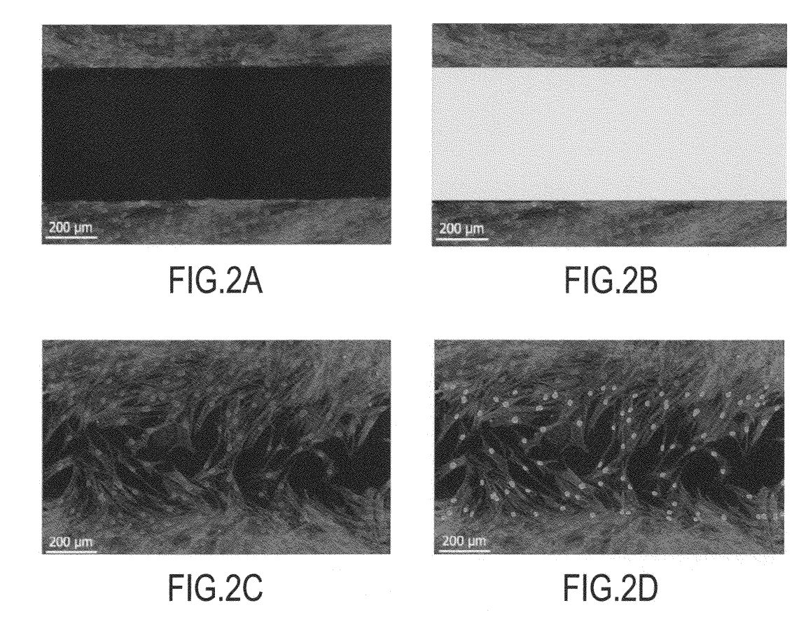

1—capture of an image before treatment (time 0), using the Nikon TE2000 video microscope equipped with the NIS software (FIG. 2a): the image shows the stripped zone, devoid of cells, after removal of the insert;

2—determination of the measurement zone by means of a computer tool-zone in yellow (FIG. 2b);

3—capture of a new image after the steps of treatment and labelling of the cell nuclei (FIGS. 2c and 2d): the damaged zone comprises fibroblasts which recolonize the initially cell-free zone;

4—automatic selection of the cell nuclei labelled in the zone defined in step 2, and counting of said labelled cell nuclei.

The number of cells that have recolonized the damaged zone, per unit area (cm²), is calculated. The measurement is reproduced on 4 images per active agents tested.

It should be noted here that FIGS. 1a, 1b, 2a and 2b were not obtained according to the treatments of Example 1, but serve to illustrate the type of photographs obtained according to the treatments of Example 1. Thus, photographs similar to those of FIGS. 1a, 1b, 2a and 2b were obtained according to the treatments of Example 1.

3. Results

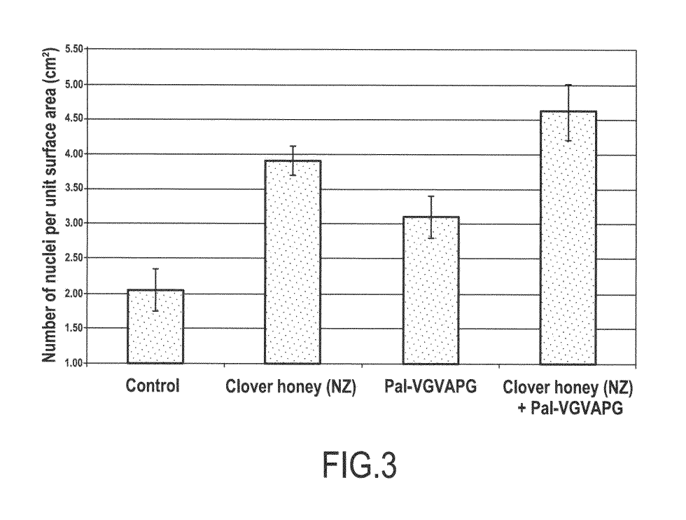

The effect of the honey tested on the recolonization of the wound is presented in FIG. 3.

Clover honey (+190% compared with the nontreated control) and Pal-VGVAPG (+150% compared with the nontreated control) have a significant effect on recolonization of the damaged zone. The combination of clover honey and Pal-VGVAPG (+224% compared with the nontreated control) exhibits an effect that is significantly greater than that observed for the treatments with clover honey or Pal-VGVAPG alone. The combination of honey and the Pal-VGVAPG peptide makes it possible to potentiate the action of each of these two active agents, which constitutes an unexpected result.

This justifies the selection of clover honey as a cosmetic active agent that is particularly effective for preventing or delaying the appearance of the signs of skin aging or slowing down the effects thereof.

### Example 2

### Cosmetic Formulations According to the Invention

The compositions below represent examples of embodiment of the invention. The percentages are expressed by weight relative to the weight of the final composition.

Antiwrinkle Serum Improving Firmness of the Skin

| | |
|---|---|
| Clover honey extract | 4 |
| Royal jelly | 0.1 |
| Val-Gly-Val-Ala-Pro-Gly peptide | 0.3 |
| Centella asiatica | 0.1 |
| Vigna aconitifolia extract | 0.1 |
| Parsol MCX | 3 |
| Excipients | qs 100 |

The serum is applied in the morning to the areas of the face showing signs of aging.

Soothing Anti-Aging Repair Night Cream

| | |
|---|---|
| Clover honey glycolysate | 3 |
| Cyathea medullaris extract | 0.2 |
| Mango butter | 2 |
| Turkesterone | 0.02 |
| Lys-Val-Gly-Val-Ala-Pro-Gly peptide | 0.1 |
| Asp-Pro-Gly-Val-Gly-Val-Ser peptide | 0.1 |
| Hyaluronic acid | 1 |
| Ascorbyl glycoside | 0.4 |
| Alpha-tocopherol | 0.2 |
| Potassium glycyrrhizinate | 0.05 |
| Fragranced emulsion excipients | qs 100 |

The cream is applied to the face at bedtime.

Antiwrinkle Repair Essence

| | |
|---|---|
| Palmitoyl-Val-Gly-Val-Ala-Pro-Gly peptide | 0.2 |
| Royal jelly | 3 |
| Bertholletia excelsa extract | 0.05 |
| Malva sylvestris extract | 0.02 |
| Glycolic acid | 0.1 |
| Alcohol | 3 |
| Adenosine | 0.02 |
| Excipients | qs 100 |

The preparation is applied to the wrinkles and fine lines of the face.

Anti-Aging Foundation

| | |
|---|---|
| Clover honey glycolysate | 0.01 |
| Royal jelly | 0.02 |
| Palmitoyl-Val-Gly-Val-Ala-Pro-Gly peptide | 0.01 |
| UVA and UVB screening agents | 5 |
| Coloured excipient | qs 100 |

The preparation is applied to the face.

Correspondence for the Sequence Listing

| | | Sequence |
|---|---|---|
| Sequences | Names | listing |
| Gly-X₁-X₂-Pro-Gly | Prot 1 | SEQ ID No. 1 |
| Val-Gly-Lys-Ser-Pro-Gly | Prot 2 | SEQ ID No. 2 |
| Ile-Gly-Lys-Ser-Pro-Gly | Prot 3 | SEQ ID No. 3 |
| Pro-Gly-Gly-Val-Lys-Pro- | Prot 4 | SEQ ID No. 4 |
| Gly | | |
| Val-Gly-Val-Val-Pro-Gly | Prot 5 | SEQ ID No. 5 |
| Ile-Gly-Lys-Gly-Pro-Gly- | Prot 6 | SEQ ID No. 6 |
| Gly-Val | | |
| Val-Gly-Ala-Met-Pro-Gly | Prot 7 | SEQ ID No. 7 |
| Val-Gly-Ala-Ser-Pro-Gly | Prot 8 | SEQ ID No. 8 |
| Val-Gly-Lys-Met-Pro-Gly | Prot 9 | SEQ ID No. 9 |
| Ile-Gly-Ala-Met-Pro-Gly | Prot 10 | SEQ ID No. 10 |
| Ile-Gly-Ala-Ser-Pro-Gly | Prot 11 | SEQ ID No. 11 |
| Ile-Gly-Lys-Met-Pro-Gly | Prot 12 | SEQ ID No. 12 |
| Val-Gly-Val-Ala-Pro-Gly | Prot 13 | SEQ ID No. 13 |
| Gly-Val-Ala-Pro-Gly-Val | Prot 14 | SEQ ID No. 14 |
| Val-Gly-Val-Ala-Pro-Gly | Prot 15 | SEQ ID No. 15 |
| Gly-Asp-Ser-Pro-Gly-Asp- | Prot 16 | SEQ ID No. 16 |
| Lys | | |
| Pro-Gly-Gly-Val-Leu-Pro- | Prot 17 | SEQ ID No. 17 |
| Gly | | |
| Val-Gly-Val-Val-Pro-Gly | Prot 18 | SEQ ID No. 18 |
| Ile-Gly-Leu-Gly-Pro-Gly- | Prot 19 | SEQ ID No. 19 |
| Gly-Val | | |
| Val-Gly-Leu-Ser-Pro-Gly | Prot 20 | SEQ ID No. 20 |
| Ile-Gly-Leu-Ser-Pro-Gly | Prot 21 | SEQ ID No. 21 |
| Ile-Gly-Val-Ala-Pro-Gly | Prot 22 | SEQ ID No. 22 |
| Val-Gly-Val-Ala-Pro-Gly | Prot 23 | SEQ ID No. 23 |
| Gly-Ala-Ala-Pro-Gly | Prot 24 | SEQ ID No. 24 |
| Gly-Val-Val-Pro-Gly | Prot 25 | SEQ ID No. 25 |
| Gly-Gly-Gly-Pro-Gly | Prot 26 | SEQ ID No. 26 |
| Gly-Leu-Leu-Pro-Gly | Prot 27 | SEQ ID No. 27 |
| Gly-Ile-Ile-Pro-Gly | Prot 28 | SEQ ID No. 28 |
| Gly-Ser-Ser-Pro-Gly | Prot 29 | SEQ ID No. 29 |
| Gly-Thr-Thr-Pro-Gly | Prot 30 | SEQ ID No. 30 |
| Gly-Cys-Cys-Pro-Gly | Prot 31 | SEQ ID No. 31 |
| Gly-Met-Met-Pro-Gly | Prot 32 | SEQ ID No. 32 |
| Gly-Phe-Phe-Pro-Gly | Prot 33 | SEQ ID No. 33 |
| Gly-Tyr-Tyr-Pro-Gly | Prot 34 | SEQ ID No. 34 |
| Gly-Trp-Trp-Pro-Gly | Prot 35 | SEQ ID No. 35 |
| Gly-Asp-Asp-Pro-Gly | Prot 36 | SEQ ID No. 36 |
| Gly-Asn-Asn-Pro-Gly | Prot 37 | SEQ ID No. 37 |
| Gly-Glu-Glu-Pro-Gly | Prot 38 | SEQ ID No. 38 |
| Gly-Gln-Gln-Pro-Gly | Prot 39 | SEQ ID No. 39 |
| Gly-Arg-Arg-Pro-Gly | Prot 40 | SEQ ID No. 40 |
| Gly-His-His-Pro-Gly | Prot 41 | SEQ ID No. 41 |
| Gly-Lys-Lys-Pro-Gly | Prot 42 | SEQ ID No. 42 |
| Gly-Pro-Pro-Pro-Gly | Prot 43 | SEQ ID No. 43 |
| Gly-(3-hydroxyproline)- | Prot 44 | SEQ ID No. 44 |
| (3-hydroxyproline)-Pro-Gly | | |
| Gly-4-hydroxyproline-4- | Prot 45 | SEQ ID No. 45 |
| hydroxyproline-Pro-Gly | | |
| Gly-Gly-Gln-Gln-Pro-Gly-Leu | Prot 46 | SEQ ID No. 46 |
| Ala-Val-Gly-Val-Ala-Pro-Gly | Prot 47 | SEQ ID No. 47 |
| Ala-Val-Gly-Val-Ala-Pro-Gly- | Prot 48 | SEQ ID No. 48 |
| Leu | | |
| Val-Gly-Gly-Val-Pro-Gly | Prot 49 | SEQ ID No. 49 |
| Leu-Gly-Thr-Ile-Pro-Gly | Prot 50 | SEQ ID No. 50 |

1. Cosmetic composition comprising at least one pharmaceutically acceptable excipient, wherein said composition comprises (i) honey or royal jelly, and (ii) a peptide, or a derivative of said peptide, comprising the sequence Gly-X₁-X₂-Pro-Gly, wherein X₁ and X₂ are amino acids selected from natural or synthetic amino acids, and derivatives thereof.

2. Composition according to claim 1, wherein the peptide comprises from 5 to 8 amino acids, or a derivative of these amino acids.

3. Composition according to claim 1, wherein X₁ and X₂ are selected from valine (Val), proline (Pro), alanine (Ala), glycine (Gly), lysine (Lys), serine (Ser), aspartic acid (Asp), arginine (Arg) and isoleucine (Ile), or a salt or derivative thereof.

4. Composition according to claim 1, wherein the peptide comprises or is formed by a sequence of amino acids selected from:
Val-Gly-Lys-Ser-Pro-Gly
Ile-Gly-Lys-Ser-Pro-Gly
Pro-Gly-Gly-Val-Lys-Pro-Gly
Val-Gly-Val-Val-Pro-Gly
Ile-Gly-Lys-Gly-Pro-Gly-Gly-Val
Val-Gly-Ala-Met-Pro-Gly
Val-Gly-Ala-Ser-Pro-Gly
Val-Gly-Lys-Met-Pro-Gly
Ile-Gly-Ala-Met-Pro-Gly
Ile-Gly-Ala-Ser-Pro-Gly
Ile-Gly-Lys-Met-Pro-Gly
Val-Gly-Val-Ala-Pro-Gly
Gly-Val-Ala-Pro-Gly-Val
Val-Gly-Val-Ala-Pro-Gly
Gly-Asp-Ser-Pro-Gly-Asp-Lys
Pro-Gly-Gly-Val-Leu-Pro-Gly
Val-Gly-Val-Val-Pro-Gly
Ile-Gly-Leu-Gly-Pro-Gly-Gly-Val
Val-Gly-Leu-Ser-Pro-Gly
Ile-Gly-Leu-Ser-Pro-Gly
Ile-Gly-Val-Ala-Pro-Gly
Val-Gly-Val-Ala-Pro-Gly
Gly-Ala-Ala-Pro-Gly
Gly-Val-Val-Pro-Gly
Gly-Gly-Gly-Pro-Gly
Gly-Leu-Leu-Pro-Gly
Gly-Ile-Ile-Pro-Gly
Gly-Ser-Ser-Pro-Gly
Gly-Thr-Thr-Pro-Gly
Gly-Cys-Cys-Pro-Gly
Gly-Met-Met-Pro-Gly
Gly-Phe-Phe-Pro-Gly
Gly-Tyr-Tyr-Pro-Gly
Gly-Trp-Trp-Pro-Gly
Gly-Asp-Asp-Pro-Gly
Gly-Asn-Asn-Pro-Gly
Gly-Glu-Glu-Pro-Gly
Gly-Gln-Gln-Pro-Gly
Gly-Arg-Arg-Pro-Gly
Gly-His-His-Pro-Gly
Gly-Lys-Lys-Pro-Gly
Gly-Pro-Pro-Pro-Gly
Gly-(3-hydroxyproline)-(3-hydroxyproline)-Pro-Gly
Gly-4-hydroxyproline-4-hydroxyproline-Pro-Gly
Gly-Gly-Gln-Gln-Pro-Gly-Leu
Ala-Val-Gly-Val-Ala-Pro-Gly
Ala-Val-Gly-Val-Ala-Pro-Gly-Leu
Val-Gly-Gly-Val-Pro-Gly
Leu-Gly-Thr-Ile-Pro-Gly.

5. Composition according to claim 1, wherein the peptide is esterified with at least one linear or branched, saturated or unsaturated, hydroxylated or nonhydroxylated, sulphur-containing or non-sulphur-containing fatty acid comprising from 2 to 22 carbon atoms.

6. Composition according to claim 1, wherein the peptide is esterified with a fatty acid comprising between 8 and 22 carbon atoms selected from a palmitoyl, stearoyl, elaidoyl, lauroyl, myristoyl, stearoyl, oleoyl, arachidyl or linoleoyl group.

7. Composition according to claim 1, wherein the peptide is a peptide comprising or formed by the sequence Val-Gly-Val-Ala-Pro-Gly, or a cosmetically acceptable salt, said polypeptide also being optionally esterified with a palmitoyl or stearoyl group, or a cosmetically acceptable salt thereof.

8. Composition according to claim 1, wherein the peptide is palmitoyl-Val-Gly-Val-Ala-Pro-Gly optionally in the form of an aqueous-glycolic solution.

9. Composition according to claim 1, wherein said honey is a unifloral or polyfloral honey.

10. Composition according to claim 1, wherein said honey is selected from the group consisting of a clover (Trifolium repens) honey, a thyme (Thymus vulgaris) honey and a manuka (Leptospernum scoparium) honey.

11. Composition according to claim 1, comprising at least one cosmetically acceptable excipient selected from pigments, dyes, polymers, surfactants, rheology agents, fragrances, electrolytes, pH adjusters, antioxidants and preservatives, and mixtures thereof.

12. Composition according to claim 1, wherein said composition is in the form of a serum, a lotion, a cream, a hydrogel a mask, a stick, or a patch.

13. Composition according to claim 1, wherein the concentration of honey or royal jelly which is used as one of the two components is between 0.001% and 10% by weight relative to the weight of the cosmetic composition containing the same.

14. Composition according to claim 1, wherein the concentration of honey or royal jelly which is used as one of the two components is between 0.01% and 5% by weight of said cosmetic composition.

15. Composition according to claim 1, wherein the concentration of abovementioned peptide or peptide derivative is between 0.001% and 5% by weight of the cosmetic composition containing same.

16. Composition according to claim 1, wherein the concentration of abovementioned peptide or peptide derivative is between 0.01% and 5% by weight of said cosmetic composition.

17. Cosmetic care method, comprising applying, to at least one area of skin, a combination of honey or royal jelly, and a peptide, comprising at least the amino acid sequence Gly-X₁-X₂-Pro-Gly, wherein X₁ and X₂ are amino acids selected from natural or synthetic amino acids, and derivatives thereof.

18. A cosmetic care method for delaying the appearance of the signs of skin aging, or slowing down the effects thereof, said method comprising applying to at least one area of skin, a combination of honey or royal jelly, and a peptide, comprising at least the amino acid sequence Gly-X₁-X₂-Pro-Gly, wherein X₁ and X₂ are amino acids selected from natural or synthetic amino acids, and derivatives thereof.


SEQUENCE LISTING (50 sequences)

SEQ ID NO: 1 - protein, 5 aa, Artificial Sequence
  Synthetic Peptide
GXXPG

SEQ ID NO: 2 - protein, 6 aa, Artificial Sequence
  Synthetic Peptide
VGKSPG

SEQ ID NO: 3 - protein, 6 aa, Artificial Sequence
  Synthetic Peptide
IGKSPG

SEQ ID NO: 4 - protein, 7 aa, Artificial Sequence
  Synthetic Peptide
PGGVKPG

SEQ ID NO: 5 - protein, 6 aa, Artificial Sequence
  Synthetic Peptide
VGVVPG

SEQ ID NO: 6 - protein, 8 aa, Artificial Sequence
  Synthetic Peptide
IGKGPGGV

SEQ ID NO: 7 - protein, 6 aa, Artificial Sequence
  Synthetic Peptide
VGAMPG

SEQ ID NO: 8 - protein, 6 aa, Artificial Sequence
  Synthetic Peptide
VGASPG

SEQ ID NO: 9 - protein, 6 aa, Artificial Sequence
  Synthetic Peptide
VGKMPG

SEQ ID NO: 10 - protein, 6 aa, Artificial Sequence
  Synthetic Peptide
IGAMPG

SEQ ID NO: 11 - protein, 6 aa, Artificial Sequence
  Synthetic Peptide
IGASPG

SEQ ID NO: 12 - protein, 6 aa, Artificial Sequence
  Synthetic Peptide
IGKMPG

SEQ ID NO: 13 - protein, 6 aa, Artificial Sequence
  Synthetic Peptide
VGVAPG

SEQ ID NO: 14 - protein, 6 aa, Artificial Sequence
  Synthetic Peptide
GVAPGV

SEQ ID NO: 15 - protein, 6 aa, Artificial Sequence
  Synthetic Peptide
VGVAPG

SEQ ID NO: 16 - protein, 7 aa, Artificial Sequence
  Synthetic Peptide
GDSPGDK

SEQ ID NO: 17 - protein, 7 aa, Artificial Sequence
  Synthetic Peptide
PGGVLPG

SEQ ID NO: 18 - protein, 6 aa, Artificial Sequence
  Synthetic Peptide
VGVVPG

SEQ ID NO: 19 - protein, 8 aa, Artificial Sequence
  Synthetic Peptide
IGLGPGGV

SEQ ID NO: 20 - protein, 6 aa, Artificial Sequence
  Synthetic Peptide
VGLSPG

SEQ ID NO: 21 - protein, 6 aa, Artificial Sequence
  Synthetic Peptide
IGLSPG

SEQ ID NO: 22 - protein, 6 aa, Artificial Sequence
  Synthetic Peptide
IGVAPG

SEQ ID NO: 23 - protein, 6 aa, Artificial Sequence
  Synthetic Peptide
VGVAPG

SEQ ID NO: 24 - protein, 5 aa, Artificial Sequence
  Synthetic Peptide
GAAPG

SEQ ID NO: 25 - protein, 5 aa, Artificial Sequence
  Synthetic Peptide
GVVPG

SEQ ID NO: 26 - protein, 5 aa, Artificial Sequence
  Synthetic Peptide
GGGPG

SEQ ID NO: 27 - protein, 5 aa, Artificial Sequence
  Synthetic Peptide
GLLPG

SEQ ID NO: 28 - protein, 5 aa, Artificial Sequence
  Synthetic Peptide
GIIPG

SEQ ID NO: 29 - protein, 5 aa, Artificial Sequence
  Synthetic Peptide
GSSPG

SEQ ID NO: 30 - protein, 5 aa, Artificial Sequence
  Synthetic Peptide
GTTPG

SEQ ID NO: 31 - protein, 5 aa, Artificial Sequence
  Synthetic Peptide
GCCPG

SEQ ID NO: 32 - protein, 5 aa, Artificial Sequence
  Synthetic Peptide
GMMPG

SEQ ID NO: 33 - protein, 5 aa, Artificial Sequence
  Synthetic Peptide
GFFPG

SEQ ID NO: 34 - protein, 5 aa, Artificial Sequence
  Synthetic Peptide
GYYPG

SEQ ID NO: 35 - protein, 5 aa, Artificial Sequence
  Synthetic Peptide
GWWPG

SEQ ID NO: 36 - protein, 5 aa, Artificial Sequence
  Synthetic Peptide
GDDPG

SEQ ID NO: 37 - protein, 5 aa, Artificial Sequence
  Synthetic Peptide
GNNPG

SEQ ID NO: 38 - protein, 5 aa, Artificial Sequence
  Synthetic Peptide
GEEPG

SEQ ID NO: 39 - protein, 5 aa, Artificial Sequence
  Synthetic Peptide
GQQPG

SEQ ID NO: 40 - protein, 5 aa, Artificial Sequence
  Synthetic Peptide
GRRPG

SEQ ID NO: 41 - protein, 5 aa, Artificial Sequence
  Synthetic Peptide
GHHPG

SEQ ID NO: 42 - protein, 5 aa, Artificial Sequence
  Synthetic Peptide
GKKPG

SEQ ID NO: 43 - protein, 5 aa, Artificial Sequence
  Synthetic Peptide
GPPPG

SEQ ID NO: 44 - protein, 5 aa, Artificial Sequence
  Synthetic Peptide
GPPPG

SEQ ID NO: 45 - protein, 5 aa, Artificial Sequence
  Synthetic Peptide
GPPPG

SEQ ID NO: 46 - protein, 7 aa, Artificial Sequence
  Synthetic Peptide
GGQQPGL

SEQ ID NO: 47 - protein, 7 aa, Artificial Sequence
  Synthetic Peptide
AVGVAPG

SEQ ID NO: 48 - protein, 8 aa, Artificial Sequence
  Synthetic Peptide
AVGVAPGL

SEQ ID NO: 49 - protein, 6 aa, Artificial Sequence
  Synthetic Peptide
VGGVPG

SEQ ID NO: 50 - protein, 6 aa, Artificial Sequence
  Synthetic Peptide
LGTIPG